(19) United States
(12) Patent Application Publication
(10) Pub. No.: US 2016/0297870 A1
(43) Pub. Date: Oct. 13, 2016

(54) Lyophilization of Hemoglobin Solutions

(71) Applicant: Enrico Bucci, Bel Air, MD (US)

(72) Inventor: Enrico Bucci, Bel Air, MD (US)

(21) Appl. No.: 14/682,396

(22) Filed: Apr. 9, 2015

(51) Int. Cl. C07K 14/805

(52) U.S. Cl. CPC C07K 14/805; A61K 38/00

(57) ABSTRACT

A lyophilized hemoglobin composition and a method for lyophilizing hemoglobin are described. The method of the invention produces a reconstituted hemoglobin solution which is not altered in functional properties from the hemoglobin solution prior to lyophilization.

This invention was funded by the National Institutes of Health. The Government has certain rights in the invention pursuant to NIH grant No. PO1-HL48517.

### INTRODUCTION

Lyophilization, or freeze-drying, of hemoglobin is useful to reduce volume and remove the need for refrigeration when hemoglobin is stored and transported for blood transfusions. There is no commonly used method of drying hemoglobin for storage and transport because known lyophilization methods degrade hemoglobin. Some currently known methods for preservation of hemoglobin either save hemoglobin in liquid form, or attempt to lyophilize entire red cells into a dry material. Disaccharides such as sucrose or various kinds of starch, or other soluble polymers are used as stabilizing agents, but none of these methods preserve hemoglobin as a soluble powder, which would totally redissolve into a native hemoglobin solution identical to the starting material (U.S. Pat. No. 5,750,330; U.S. Pat. No. 6,242,417; U.S. Pat. No. 5,690,963; U.S. Pat. No. 5,929,031). Thus a need exists for a method of hemoglobin lyophilization that allows storage and shipping of hemoglobin, including transport into remote environments without refrigeration.

### SUMMARY OF THE INVENTION

The present invention provides for a method for lyophilizing hemoglobin. The method includes dialyzing the hemoglobin solution against a solution containing a stabilizing agent or adding a stabilizing agent to the hemoglobin solution prior to lyophilization. The solid hemoglobin powder obtained by lyophilizing the hemoglobin compositions of the present invention having a stabilizing agent provides the best storage technology for hemoglobin compounds.

Therefore, it is an object of the present invention to provide novel hemoglobin solutions for lyophilization wherein the hemoglobin solutions have been mixed with a stabilizing agent. The hemoglobin solutions can be dialyzed against a stabilizing agent or the stabilizing agent can be added to a hemoglobin solution to make a hemoglobin-stabilizing agent solution.

The stabilizing agent of the present invention can be a monosaccharide, a disaccharide, a polysaccharide, or a soluble polymer, or a combination of stabilizing agents.

It is yet another object of the present invention to provide a method for lyophilizing a hemoglobin comprising:

dialyzing a hemoglobin solution against a solution containing a stabilizing agent in a buffer; and

lyophilizing said dialyzed hemoglobin solution.

It is another object of the present invention to provide a method for lyophilizing a hemoglobin comprising:

adding a stabilizing agent to hemoglobin in solution; and

lyophilizing said stabilizing agent-hemoglobin solution.

It is still another object of the present invention to provide lyophilized hemoglobin for transport and storage. The lyophilized hemoglobin solution of the present invention is easy to reconstitute and is ready for use as an in vivo infusible fluid.

It is another object of the present invention to provide a method for reconstituting lyophilized hemoglobin comprising adding a buffer solution to produce a fluid with proper osmolarity for in vivo use made by the preexisting stabilizing agent in the lyophilized hemoglobin and the saline content of the buffer.

It is yet another object of the present invention to provide reconstituted lyophilized hemoglobin for regular use either for in vitro experiments or for in vivo infusible fluids.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1. Elution profile through sepharose gel of ZL-HbBv polymer (described below as a polymerized form of bovine hemoglobin) in 0.01 M tris-acetate buffer at pH 8.3, at room temperature before lyophilization (solid line) and after lyophilization (dotted line). Data not normalized. Results indicate that no low molecular weight species are formed during lyophilization. The small trailing peak is probably an artefact of the chromatography. The average molecular size of the main fraction is near 25 mega Dalton.

FIG. 2. Superimposition of the visible absorption spectra of Carbonmonoxy-HbA before (solid line) and after (dotted line) lyophilization. The lyophilization procedure did not produce ferric forms of the protein. In 0.01 M tris-acetate buffer at pH 8.3 at room termperature.

FIG. 3. Superimposition of ZL-HbBv absorption spectra in the oxygenated form, before (solid line) and after (dotted line) lyophilization. The spectra are totally superimposable indicating that no ferric forms were produced by the lyophilization/reconstitution procedure. In 0.01 M tris-acetate buffer at pH 8.3 at room temperature.

FIG. 4. Superimposition of the raw data of the oxygen binding curves of HbA before (solid line) and after (dotted line) lyophilization. The respective P₅₀ were 9.44 and 9.13 mmHg. The cooperativity indexes “n” were 2.72 and 2.45 respectively. In 0.01 M tris-acetate buffer at pH 8.3 at room temperature.

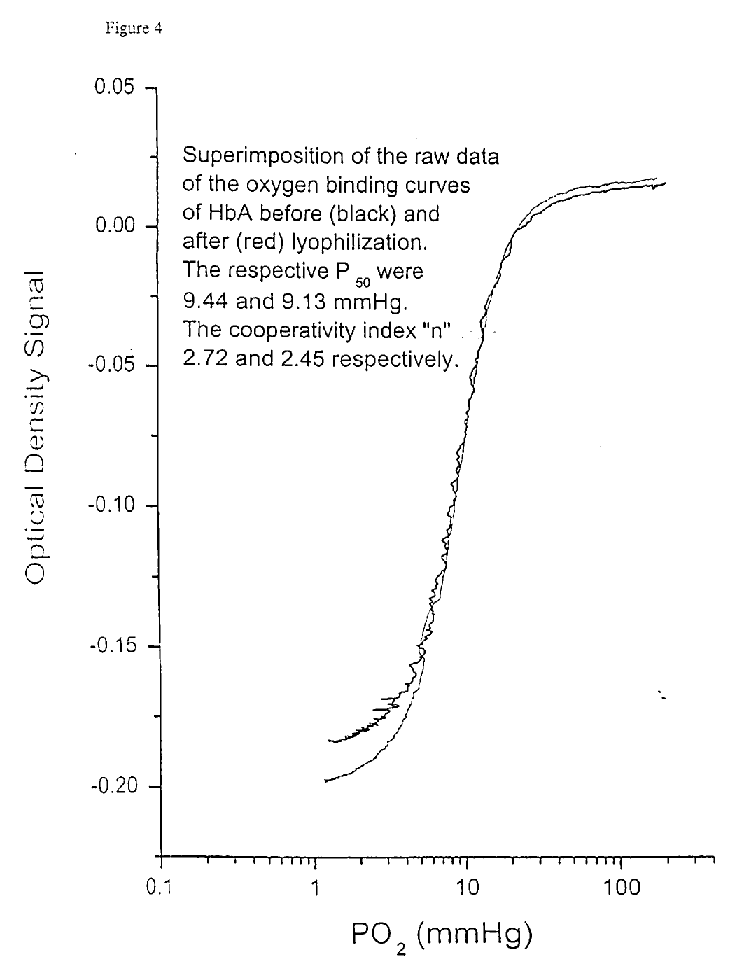

FIG. 5. Superimposition of the Raw Data of the oxygen binding curves of ZL-HbBv before (solid line) and after (dotted line) lyophilization. The slight difference reflects a different protein concentration of the samples. There is no oxygen binding cooperativity. The respective P₅₀ were 4.0 and 3.7 mmHg. In 0.01 M tris-acetate buffer at pH 8.3 at room temperature.

### DETAILED DESCRIPTION

As used herein, the terms below and variations thereof shall be defined as follows, unless otherwise indicated:

By “osmolarity” is meant the osmotic activity expressed in terms of osmoles or milliosmoles of salt solution. When hemoglobin is present in solution the osmotic activity is reported per gram of protein in salt solution.

Hemoglobin—Hemoglobin (Hb) is the oxygen-carrying component of blood that circulates through the bloodstream inside small enucleate cells known as erythrocytes or red blood cells. It is a protein comprised of four associated polypeptide chains that bear prosthetic groups known as hemes. The structure of hemoglobin is well known and described in Bunn & Forget, eds., Hemoglobin: Molecular, Genetic and Clinical Aspects (W. B. Saunders Co., Philadelphia, Pa.: 1986) and Fermi & Perutz “Hemoglobin and Myoglobin,” in Phillips and Richards, Atlas of Molecular Structures in Biology (Clarendon Press: 1981). As blood circulates through the lungs, the oxygen present in the alveolar capillaries diffuses through the alveolar membrane and acts to convert virtually all of the hemoglobin within the red cells to a reversible molecular complex known as oxyhemoglobin. During this oxygenation process, the red blood cells become cherry red in color. Because the association of the oxygen and hemoglobin molecules within the red cells is reversible, the oxygen molecules are gradually released from the hemoglobin molecules (or from the red blood cells) when blood reaches the tissue capillaries. Eventually, the oxygen molecules diffuse into the tissues and is consumed by metabolism. As the oxyhemoglobin reduces to hemoglobin, the red cells become purple in color. Hemoglobin as used herein refers to all normal and mutant native vertebrate, mammalian, including human hemoglobins obtained either from drawn blood or by recombinant procedures, their chemically treated or surface decorated derivative either in their dimeric, or tetrameric or variously polymerized forms.

Expression of various recombinant hemoglobins has been achieved. Such expression methods include individual globin expression as described, for example, in U.S. Pat. No. 5,028,588, and di-alpha globin expression created by joining two alpha globins with a glycine linker through genetic fusion coupled with expression of a single beta globin gene to produce a pseudotetrameric hemoglobin molecule as described in WO 90/13645 and Looker et al., Nature 356:258 260 (1992). Other modified recombinant hemoglobins are disclosed in PCT Publication WO 96/40920. Similar to other heterologous proteins expressed in E. coli, recombinant hemoglobins have N-terminal methionines, which in some recombinant hemoglobins replace the native N-terminal valines. For products at present in clinical trials, raw hemoglobin is obtained either from outdated human banked blood, cows, or recombinant (E. coli) sources. Estimates are that one percent or less of stored blood becomes outdated, making only about 120,000 units of blood available for the manufacture of blood substitutes annually. Although not reported, the yield of these finished infusible products, after the necessary chemical manipulations, can be estimated at 30% of the initial material. The current procedures for preparing native hemoglobin solution from human blood involve extensive process of washing pooled red cells with saline, sedimentation or diafiltration, gentle lysis with hypotonic buffers, and rigorous removal of red cell membranes.

The hemoglobin solution at whatever concentration desired to be lyophilized, is dialyzed against a solution containing a stablilizing agent or stabilizing agent is added to the hemoglobin solution. By “stabilizing agent” is meant saccharide, disaccharide, polysaccharide, and soluble polymer. By monosaccharide is meant a saccharide which may include, but not be limited to, glucose, fructose, galactose, ribose, mannitol, sorbitol or xylitol or a mixture thereof. Disaccharides include, but are not limited to, sucrose, trehalose, and raffinose, more preferably trehalose. As said polysaccharides are polymers of monosaccharides joined by glycosidic linkages. Starches are glucose polymers, and of the many available starches, potato starch is the most preferred. As examples of potato starches mention can be made of Pharma M20. Starch can be extracted from a variety of vegetables and greatly vary in molecular size, ranging from about 5 to about 20 kDa, or more. Their solubility is inversely proportional to their size. Efficacy of the agent is determined by comparing the physicao-chemical and functional properties of hemoglobin before and after dialysis. A “water-soluble polymer” as defined herein is any polymer which is soluble in water or an aqueous-based system. Water-soluble polymers suitable for use in the invention include water-soluble polysaccharides, for example, ficoll and polymer surfactants, in particular, nonionic polymer surfactants. Suitable nonionic polymer surfactants include poloxamers, which are polyethylenepolypropyleneglycol polymers commonly referred to as Pluronics. For example poloxamer 407 sold under the trademark PLURONIC F127, poloxamer 188 sold under the trademark PLURONIC F68 (available from BASF Wyandotte) and combinations thereof. Polysorbates are another type of nonionic surfactant often referred to as polyoxyethylene sorbitan esters. Polysorbate 80 sold under the trademark TWEEN® 80, polysorbate 20 sold under the trademark TWEEN® 20 and combinations thereof are suitable polysorbates for use as the water soluble polymer of the invention. Other water-soluble nonionic polymer surfactants suitable for use in the invention include, but are not limited to, polyethylene glycol polymers, polyvinylpyrrolidones, and any combinations of any of the above. Combinations of agents may be used knowing that solubility is reduced in a crowded environment especially when polymers are used.

Alternatively, the stabilizing agents can be added to the hemoglobin solutions. Other stabilizing agents can be used as long as they are not toxic. The stabilizing agent is present in the dialyzing solution in an amount of between 20-80 mg/ml, preferably 30-70 mg/ml, and more preferably 55 mg/ml.

In addition, buffers are present in the dialysis solution. The pH should preferably be maintained in the range of between 7.5 and 9.0 as measured at room temperature, during lyophilization, and more preferably at a pH of about 8.3. The buffering agent can be any physiologically acceptable chemical entity or combination of chemical entities, which in the absence of chloride ions, have the capacity to act as pH buffers, including, Tris-acetate, Tris, BIS-Tris Propane, PIPES, MOPS, HEPES, MES and ACES. The full chemical designations of these buffering agents is listed in Table 1 below. Typically, the buffering agent is included in a concentration of 5-20 mM, more preferably around 10 mM. Tris-acetate is especially preferred as the buffering agent.

TABLE 1
Buffering Agents

Tris tris-(hydroxymethyl)-aminomethane
BIS-Tris Propane 1,3-bis-[tris-(hydroxy-
methyl)methylamino]-propane
PIPES piperazine-N,N′-bis-(2-ethanesulfonic acid)
MOPS 3-{N-morpholino) propanesulfonic acid
HEPES N-2-hydroxyethyl-piperazine-N′-2-
ethanesulfonic acid
MES 2-(N-morpholino) ethanesulfonic acid
ACES N-2-acetamido-2-aminoethanesulfonic acid

NADH, the oxidized form of nicotinamide adenine dinucleotide is optionally included in the present dialysis solution in an amount of 2-7 mM, preferably 3-6 mM, and most preferably about 5 mM, to prevent hemes oxidation to their ferric form, if necessary.

Various technologies can be used for transferring hemoglobins from one solvent to another such as diafiltration, gel filtration, fixed volume dialysis, semipermeable dialysis tubes. When semipermeable dialysis tubings are used, dialysis should last at least 4 hours, preferably overnight, or until the pH of the new buffer is reached.

These hemoglobin solutions are then lyophilized by freezing the solutions at −20° C. or less and kept under vacuum with the help of a high vacuum pump, mechanical or cryo-pumps. The end product is a powder, which redissolves completely, without insoluble residuals. The powder can be stored in bottles or vials of glass or plastic, preferably glass.

The hemoglobin used in the present formulations can be either blood-derived hemoglobin or recombinantly produced hemoglobin. The blood-derived hemoglobin may be from blood of any vertebrate.

For usage as infusable fluid in vivo after lyophilization, and if trehalose concentration was 50 mg/ml, the lyophilized hemoglobin can be reconstituted to the original volume with a saline solution at about 150 milliosmolar (mosm), for example with half diluted commercially available Ringer's solution (Baxter, Deer Park, Ill.), or any other saline solution with osmolarity near 150 mosm, such that, when reconstituted, the final osmolarity of the solution is about 300 mosm, resulting from the trehalose content and the saline content. In this way, the reconstituted solution is ready for infusion, i.e. the osmolality is physiologic and trehalose is a non toxic sugar easily metabolized.

In order to achieve maximal stability, the hemoglobin compositions of the present invention are preferably lyophilized. During lyophilization, hemoglobin is converted into a powder form, which can be stored for weeks at room temperature or in normal refrigerators at 5° C. or stored for years at −20° C., in a freezer, or indefinitely in a deep freezer at −120° C. Mail shipments do not require refrigeration, and plastic bags can be used for mailing, which greatly simplifies packaging. This is a big advantage of lyophilization over any other form of hemoglobin storage in liquid form. The powder obtained with methods of the present invention does not contain ferric forms of hemoglobin at higher concentrations than those before lyophilization. Also, the powder redissolves completely, without any residual.

Further information on lyophilization may be found in Carpenter, J. F. and Chang, B. S., Lyophilization of Protein Pharmaceuticals, Biotechnology and Biopharmaceutical Manufacturing, Processing and Preservation, K. E. Avis and V. L. Wu, eds. (Buffalo Grove, Ill.: Interpharm Press, Inc.), pp. 199 264 (1996).

All publications, including, but not limited to patents and patent applications, cited in this specification are herein incorporated by reference as if each individual publication were specifically and individually indicated to be incorporated by reference herein as though fully set forth.

The invention is further described in detail to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided therein.

The following Materials and Methods were used in lyophilization experiments, the results of which are found in the Brief Description of the Drawings above.

Materials and Methods

Hemoglobin A was prepared by chromatographic procedures as described in Bucci, E. Biophysical Cemistry 97:103, 1973.

Zl-HbBv, i.e. zero link polymerized bovine hemoglobin was prepared by treating bovine hemiglobin solution with 1-ethyl-3-(3-dimethylaminoprppyl)carbodiimide (EDC), as described in Matheson et al. J. Appl. Physiol. 93:1479-1483, 2002. Briefly, bovine oxyhemoglobin is first crosslinked with 3,5,bis(dibromosalicyl)adipate, following by polymerization with EDC, pasteurization, reduction of the ferric forms so produced, detoxification, storage in liquid or powder form.

Lyophilization was performed with equipment HETO, Type CT110 by Appropriate Technical Resources (ATR, Laurel, Md.)), using the protocol recommended by manufacturer.

Spectrophotomety was performed using a Hewlett Packard array spectrophotometer Hewlett Packard 8452 Diode Array spectrophotometer (Palo Alto, Calif.).

Gel filtration was performed using Exclusion-HPLC; Waters 4000 System (Milford, Mass.), Fractogel (EMD Biosec EMD Industries, Darmstadt, GE), 20-40 microns particle size, detection: absorbance at 280 nm wavelength.

Oxygen binding isotherms were measured using a Hemoxanalizer (TCS, Southampton, Pa.) in 0.1M Tris pH 7.4 37° C., protein concentration 1 mg/ml.

Trehalose was obtained from Sigma Chemical Co. (St. Louis, Mo.).

### Example 1

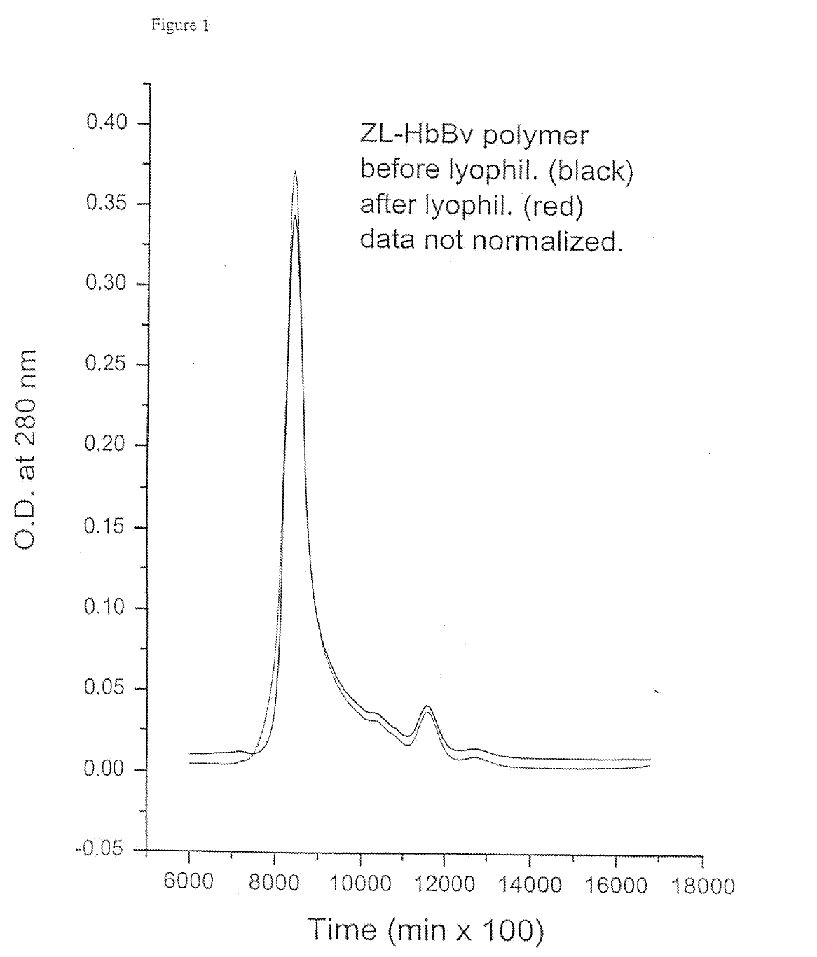

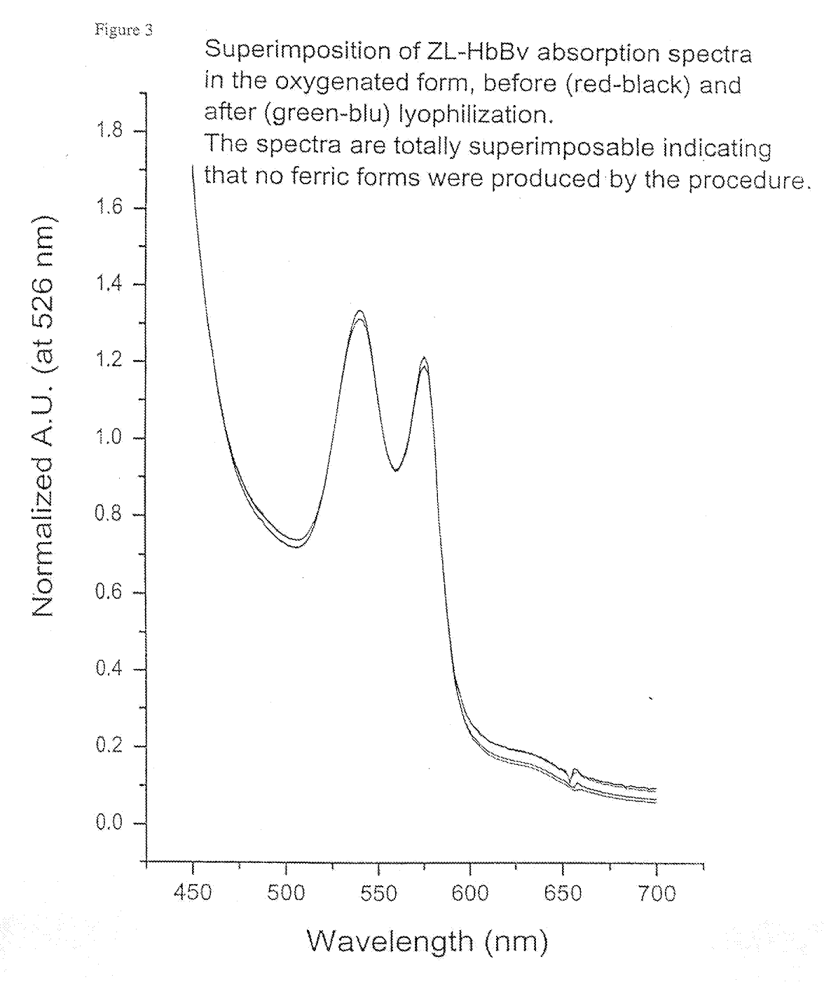

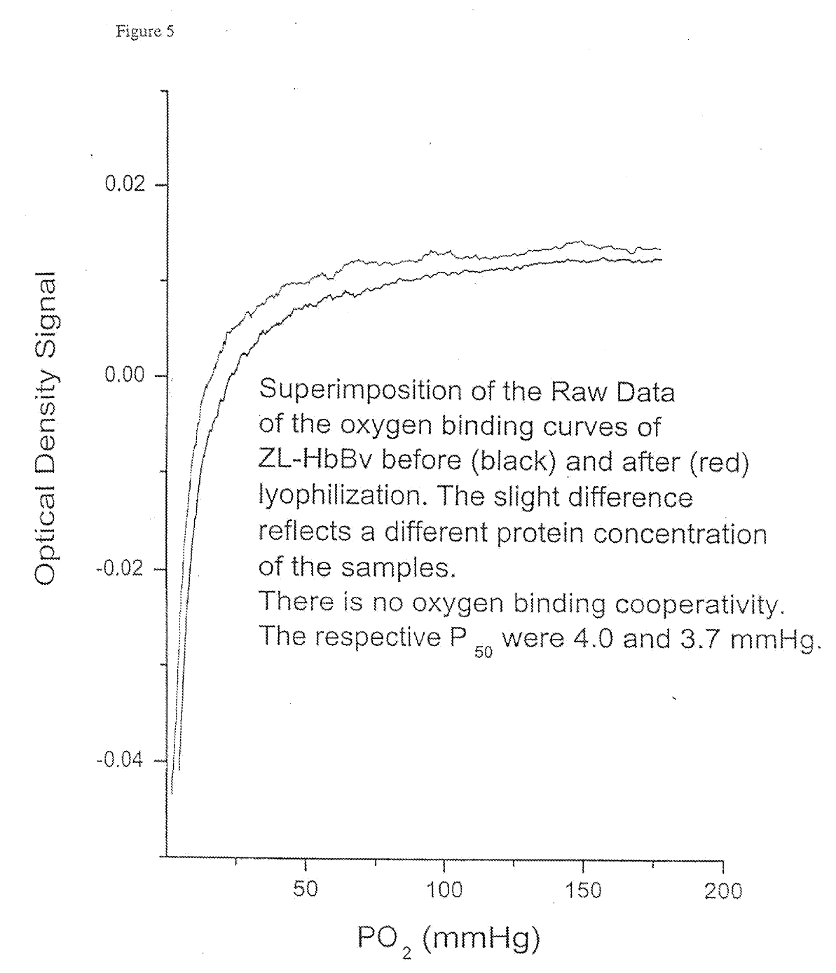

A Zl-HbBv solution was dialyzed against a 55 mg/ml trehalose in 0.01 M tris-acetate at pH8.3 and 5 mM NADH. After lyophilization, reconstitution to the original volume with half-diluted Ringer (so as to be at 150 mosm) will produce a fluid with osmolarity near 300 mosm, made by 55 mg/ml of trehalose and half the saline content of ringer. The elution profile through sepharose gel of ZL-HbBv polymer (described below as a polymerized form of bovine hemoglobin) in 0.01 M tris-acetate buffer at pH 8.3, at room temperature before lyophilization (solid line) and after lyophilization (dotted line) is shown in FIG. 1. Data not normalized. Results indicate that no low molecular weight species are formed during lyophilization. The small trailing peak is probably an artefact of the chromatography. The average molecular size of the main fraction is near 25 mega Dalton. Superimposition of ZL-HbBv absorption spectra in the oxygenated form, before (solid line) and after (dotted line) lyophilization are shown in FIG. 3. The spectra are totally superimposable indicating that no ferric forms were produced by the lyophilization/reconstitution procedure. Superimposition of the Raw Data of the oxygen binding curves of ZL-HbBv before (solid line) and after (dotted line) lyophilization is shown in FIG. 5. The slight difference reflects a different protein concentration of the samples. There is no oxygen binding cooperativity. The respective P₅₀ were 4.0 and 3.7 mmHg.

### Example 2

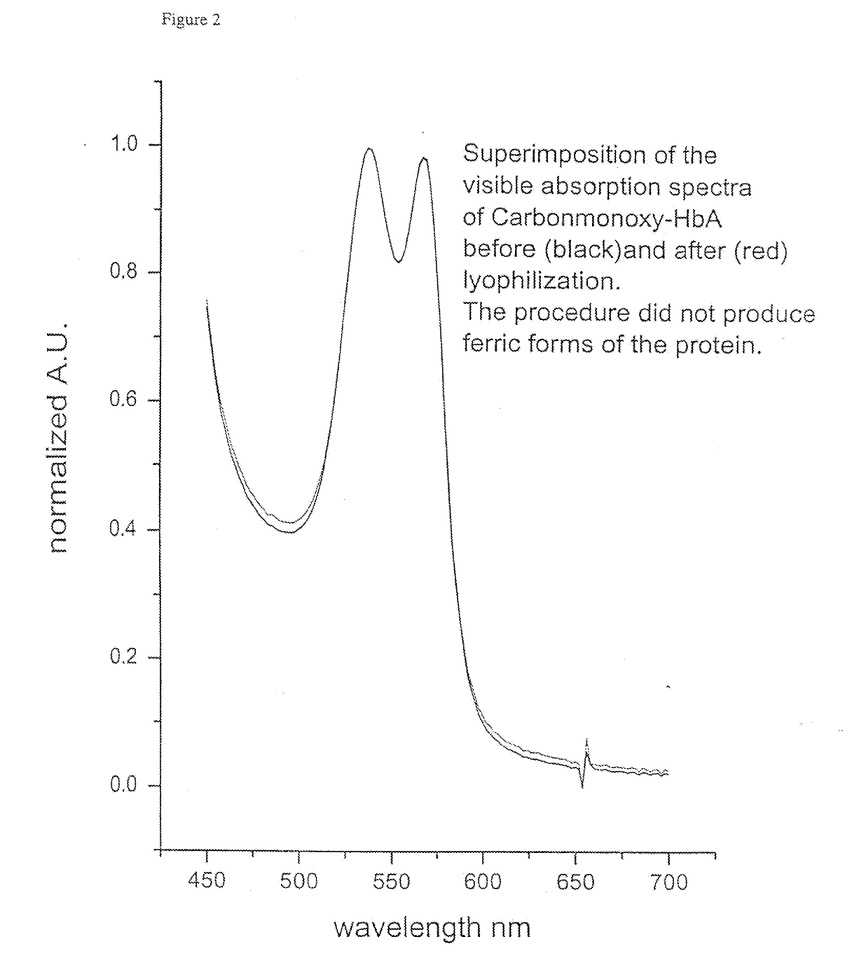

Hemoglobin A was prepared by chromatographic procedure per Materials and Methods. The hemoglobin solutions was dialyzed and the dialyzed hemoglobin solution was lyophilized and reconstituted as for ZL-HbBv in Example 1. Superimposition of the visible absorption spectra of Carbonmonoxy-HbA before (solid line) and after (dotted line) lyophilization is shown in FIG. 2. The lyophilization procedure did not produce ferric forms of the protein. Superimposition of the raw data of the oxygen binding curves of HbA before (solid line) and after (dotted line) lyophilization is shown in FIG. 3. The respective P₅₀ were 9.44 and 9.13 mmHg. The cooperativity indexes “n” were 2.72 and 2.45 respectively.

TABLE 1
Summary of the results shown in the
Figures.

| | HbA | ZL-HbBv |
|---|---|---|
| P50 before lyophilization (mmHg) | 9.44 (n = 2.72) | 4.0 |
| P50 after lyophilization (mmHg) | 9.13 (n = 2.45) | 3.7 |
| Ferric form before lyophilization % | 7.8 | 17.5 |
| Ferric form after lyophilization % | 9.1 | 17.5 |

What is claimed is:

1. A lyophilized hemoglobin composition, said composition produced by lyophilizing a hemoglobin aqueous solution comprising:
a stabilizing agent selected from the group consisting of saccharide, disaccharide, polysaccharide, and soluble polymer; and
a buffering agent.

2. The lyophilized hemoglobin composition of claim 1, wherein said disaccharide is chosen from the group consisting of sucrose, trehalose, raffinose.

3. The lyophilized hemoglobin composition of claim 2 wherein said disaccharide is trehalose.

4. The lyophilized hemoglobin composition of claim 3 wherein said trehalose is at a concentration of 20-80 mg/ml.

5. The lyophilized hemoglobin composition of claim 4, wherein said stabilizing agent is present in an amount of about 55 mg/ml.

6. The lyophilized hemoglobin composition of claim 1, wherein said buffering agent comprises between 5 mM and 20 mM Tris-acetate.

7. The lyophilized hemoglobin composition of claim 6, wherein the Tris-acetate is present in an amount of about 10 mM.

8. The lyophilized hemoglobin composition of claim 1, further comprising NADH.

9. The lyophilized hemoglobin composition of claim 8, wherein said NADH is present in an amount of about 3-6 mM.

10. The lyophilized hemoglobin composition of claim 9, wherein said NADH is present in an amount of about 5 mM.

11. The lyophilized hemoglobin composition of claim 1 in combination with a diluent.

12. The lyophilized hemoglobin composition of claim 9, wherein the diluent produces a final osmolarity of about 300 mosm.

13. A method for producing a lyophilized hemoglobin comprising
dialyzing a hemoglobin solution against a solution comprising a stabilizing agent selected from the group consisting of a saccharide, a disaccharide, a polysaccharide, and a soluble polymer and a buffering agent to produce a dialyzed hemoglobin solution; and
lyophilizing said dialyzed hemoglobin solution.

14. The method of claim 13 wherein said disaccharide is chosen from the group consisting of sucrose, trehalose, and raffinose.

15. The method of claim 14 wherein said disaccharide is at a concentration of 20-80 mg/ml.

16. The method of claim 15 wherein said disaccharide is at a concentration of 55 mg/ml.

17. The method of claim 16 wherein said disaccharide is trehalose.

18. A method for producing lyophilized hemoglobin comprising
adding to a hemoglobin solution a stabilizing agent selected from the group consisting of a saccharide, a disaccharide, a polysaccharide, and a soluble polymer to produce a hemoglobin-stabilizing agent solution.

19. The method of claim 18 wherein said disaccharide is chosen from the group consisting of sucrose, trehalose, and raffinose.

20. The method of claim 19 wherein said disaccharide is at a concentration of 20-80 mg/ml.

21. The method of claim 20 wherein said disaccharide is at a concentration of 55 mg/ml.

22. The method of claim 21 wherein said disaccharide is trehalose.
